(19) United States
(12) Patent Application Publication
(10) Pub. No.: US 2023/0149608 A1
(43) Pub. Date: May 18, 2023

(54) FLUID SENSOR SYSTEM

(71) Applicant: Analog Devices International Unlimited Company, Limerick (IE)

(72) Inventors: Youri Victorovitch Ponomarev, Rotselaar (BE); Claire E. Leahy, Listowel (IE); Liam Riordan, Patrickswell (IE); Aileen Anne Cleary, Somerville, MA (US)

(21) Appl. No.: 18/055,799

(22) Filed: Nov. 15, 2022

Related U.S. Application Data
(60) Provisional application No. 63/280,486, filed on Nov. 17, 2021.

(51) Int. Cl. A61M 1/16

(52) U.S. Cl. CPC A61M 1/1607; A61M 2205/3331; A61M 2205/70

(57) ABSTRACT

A fluid sensor system that is configured to perform in-line monitoring is disclosed. The fluid sensor system can include a sensor module that includes a sensing channel configured to receive a sample fluid, two or more calibration compartments, and a sensing element configured to interact with the sample fluid in the sensing channel. The fluid sensor system can include a reader that is electrically and mechanically coupled to the sensor module. The reader includes a controller that is configured to control operation of the fluid sensor system.

### RELATED APPLICATIONS

This application claims the benefit of U.S. Provisional Application No. 63/280,486 entitled “FLUID SENSOR SYSTEM,” filed Nov. 17, 2021, the entire disclosure of which is incorporated herein by reference for all purposes. This application also relates to U.S. application Ser. No. 17/654,177 (US Publication No. 2022/0291165), filed Mar. 9, 2022, the entire disclosure of which is incorporated herein by reference for all purposes.

### BACKGROUND

### Field

The field relates to a fluid sensor system, and self-contained in-line monitoring system of concentrations of species in physiological extracorporeal and other medical-treatment related fluidic circuits.

### Description of the Related Art

Many medical treatment procedures are performed in a hospital or outpatient facility, such that the patient must typically be admitted to the facility to undergo treatment. Treatment procedures, such as kidney dialysis procedures, may need to be performed on a regular basis, which can be inconvenient, time-consuming, and economically costly to the patient and the clinician. Enabling such treatment procedures to be performed in one location (e.g., the patient's home) can advantageously improve the convenience, efficiency, and affordability of the procedures.

### SUMMARY

For purposes of summarizing the disclosure, certain aspects, advantages, and novel features of the innovations have been described herein. It is to be understood that not necessarily all such advantages can be achieved in accordance with any particular embodiment. Thus, the innovations described herein can be embodied or carried out in a manner that achieves or optimizes one advantage or group of advantages as taught herein without necessarily achieving other advantages as can be taught or suggested herein.

In one aspect, a fluid sensor system configured to perform in-line monitoring is disclosed. The fluid sensor system can include a sensor module. The sensor module includes a sensing channel that is configured to receive a sample fluid, two or more calibration compartments, and a sensing element that is configured to interact with the sample fluid in the sensing channel. The fluid sensor system can include a reader that is electrically and mechanically coupled to the sensor module. The reader includes a controller that is configured to control operation of the fluid sensor system. The fluid sensor system is configured to connect in-line with a treatment system.

In one embodiment, the sensing element includes a plurality of transducers. The transducers can be configured to interact with the sample fluid and transmit a signal indicative of a particular constituent component of the sample fluid. The sensing element can include at least three transducers.

In one embodiment, the two or more calibration compartments includes a first calibration compartment that is configured to store a first calibration fluid. The two or more calibration compartments can include a second calibration compartment that is configured to store a second calibration fluid different from the first calibration fluid.

In one embodiment, the reader is connectible to an external device and configured to send measurement results to the external device.

In one embodiment, the fluid sensor system further includes a fluid pathway having a fluid inlet and a fluid outlet. The sensing channel can be connected to a portion of the fluid pathway between the fluid inlet and the fluid outlet. The fluid sensor system can further include a valve between the sensing channel and the fluid pathway. The controller can be configured to control the valve to open or close. An exit channel downstream of the sensing element can be connected to the fluid pathway. The fluid inlet or the fluid outlet can include a Luer lock configured to couple to a treatment system.

In one embodiment, the fluid sensor system further includes a waste compartment that is positioned downstream of and connected to the sensing element by way of an exit channel.

In one embodiment, a sample exit channel downstream of the sensing element is connected to the treatment system through a fluid outlet of the fluid sensor system.

In one aspect, a method of monitoring a sample fluid is disclosed. The method can include connecting a sensor module in-line with a treatment system, providing the sample fluid from the treatment system to a sensing element to thereby cause the sample fluid to interact with the sensing element, transmitting a signal indicative of a particular constituent component of the sample fluid by the sensing element to a reader electrically and mechanically coupled to the sensor module, and calibrating the sensing element using a calibration fluid stored in two or more calibration compartments.

In one embodiment, a fluid inlet and a fluid outlet of the sensor module connect to the treatment system by way of a Luer lock.

In one embodiment, the sensing element comprises a plurality of transducers.

In one embodiment, the calibration fluid is stored in a first calibration compartment of the two or more calibration compartments, and a second calibration fluid is stored in a second calibration compartment of the two or more calibration compartments.

In one embodiment, the sensor module includes a waste compartment positioned downstream of the sensing element and configured to receive the sample fluid that has passed through the sensing element.

In one embodiment, the method further includes calibrating the sensor module after providing the sample fluid to the sensing element. The method can further includes providing a second sample fluid from the treatment system to the sensing element to thereby interact the second sample fluid with the sensing element.

### BRIEF DESCRIPTION OF THE DRAWINGS

Specific implementations will now be described with reference to the following drawings, which are provided by way of example, and not limitation.

FIG. 1 shows a block diagram of a sensor system according to an embodiment.

FIG. 2 shows a more detailed block diagram of the sensor system according to an embodiment.

FIG. 3 is a system outline of the sensor system according to an embodiment.

FIG. 4A is a system outline of a sensor system according to another embodiment.

FIG. 4B is a system outline of a sensor system according to another embodiment.

FIG. 5A shows a timing diagram of operating a sensor module of a sensor system, according to an embodiment.

FIG. 5B is a chart showing example timings of various states in the operation of the sensor module.

FIG. 6 is a flow chart showing a method of monitoring a sample fluid.

### DETAILED DESCRIPTION

Various embodiments disclosed herein relate to a fluid sensor module configured to be connected in-line with a treatment system. The treatment system can comprise a medical device, such as a dialysis treatment system. The fluid sensor module can include a fluid inlet and a fluid outlet configured to fluidly connect to a treatment system of the medical device. For example, in kidney hemodialysis systems, blood can be transferred from the patient, through the dialysis treatment system, and back into the patient, to treat the patient's blood. In a peritoneal dialysis (PD) system, the treatment system can pump dialysate into the abdominal cavity and then remove the effluent after some dwell time. During a treatment procedure, it can be important to monitor the composition of constituent materials in a sample fluid such as a treatment fluid (e.g., dialysate) and/or the patient's blood, such as creatinine, potassium, sodium, or any other constituent material that should be monitored. The fluid sensor module can be placed upstream or downstream of the treatment system to monitor the constituent materials.

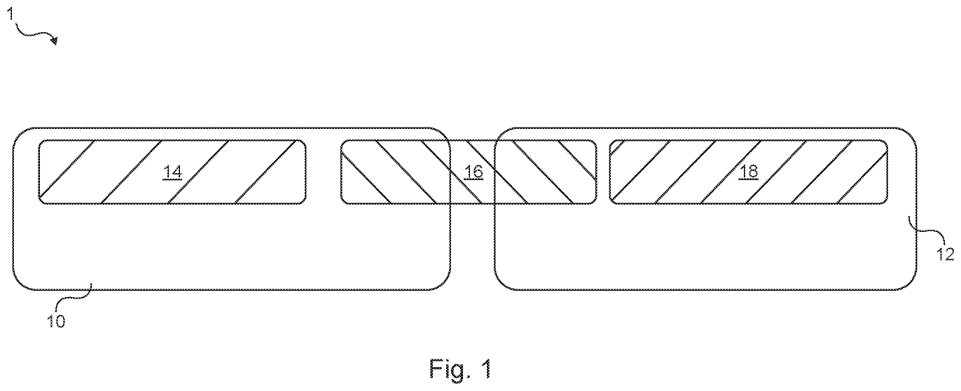

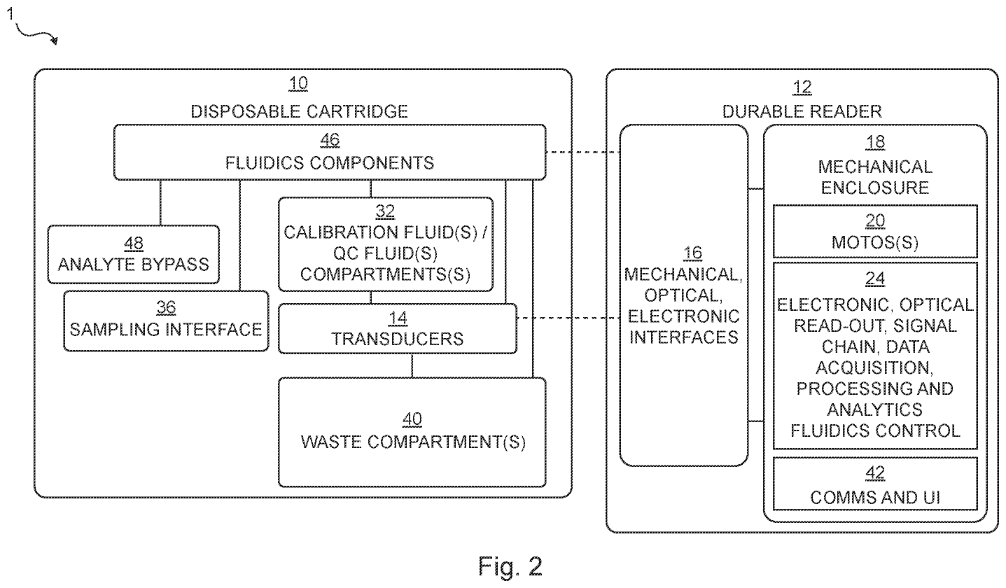

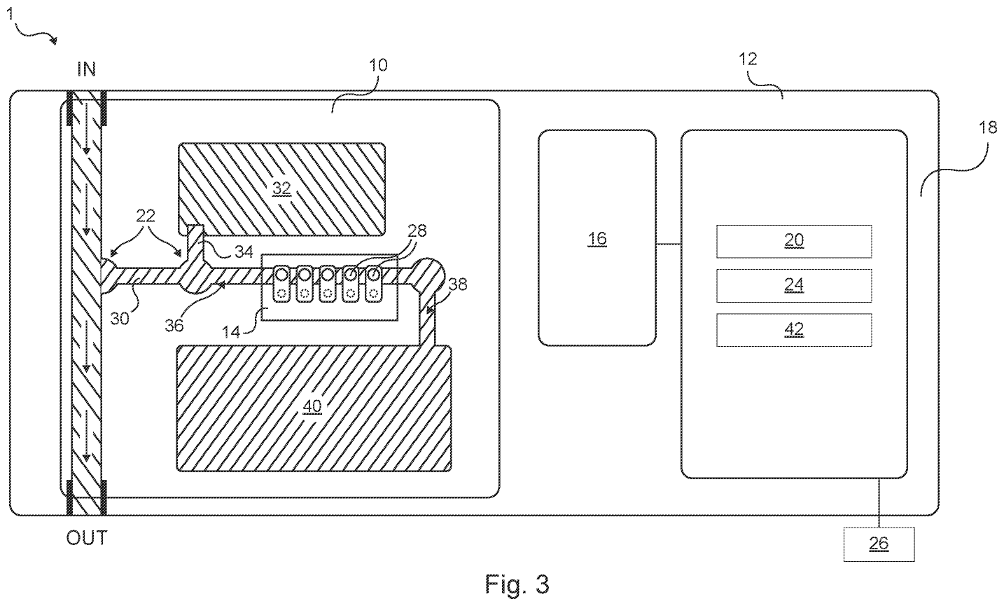

FIG. 1 shows a block diagram of a sensor system 1 according to an embodiment. FIG. 2 shows a more detailed block diagram of the sensor system 1 according to an embodiment. FIG. 3 is a system outline of the sensor system 1 according to an embodiment. In some embodiments, the sensor system 1 can comprise a fluid sensor system. The sensor system 1 can include a sensor module 10 and a reader 12. The sensor module 10 can be disposable. The sensor module 10 can also be referred to as a sensor cartridge. In some embodiments, the sensor module 10 can be removably coupled to the reader 12. The sensor module 10 can include a transducer chip 14. The sensor system 1 can include an interface unit 16. The interface unit 16 can function as an interface between the sensor module 10 and the reader 12, and be part of the sensor module 10 and/or the reader 12. The reader 12 can include a controller 18. In some embodiments, the reader 12 and the sensor module 10 can communicate through the interface unit 16.

Beneficially, the sensor module 10 (e.g., the fluid sensor module) can be sized and configured to be used at home by the patient, or in a clinical setting such as a hospital or clinic. For example, a fluid inlet (IN) and a fluid outlet (OUT) can connect to the treatment system by way of a quick connection such as a Luer lock or other fluid coupling. The sensor module 10 can mechanically and electrically connect to the reader 12. The reader 12 can comprise one or more valve motors 20 connectable to one or more valves 22 in a housing of the sensor module 10. Leads of the reader 12 can electrically connect to corresponding I/O pads of the sensor module 10. The reader 12 can comprise processing electronics 24 configured to control the operation of the valve motor 20 and/or the transducer chip 14. The processing electronics 24 can also be configured to bias, store and/or process signals transduced by the sensor module 10 and transferred to the leads by way of the I/O pads. For example, the processing electronics 24 can convert analog signals to digital signals. In various embodiments, the processing electronics 24 can be configured to identify a constituent composition of the sample fluid. The reader 12 can communicate with a computing device 26, such as a central server, a mobile device (e.g., a smartphone), a laptop computer, or the like to convey the sensed data to the clinician. For example, the reader 12 can wirelessly transmit the data (e.g., measurement results) to the computing device 26. In other embodiments, the reader 12 can be electrically connected to the computing device 26 by an electrical connector, such as a cable or cord.

The sensor module 10 can comprise a sensing assembly (e.g., a sensing element) including a plurality of bare and functionalized transducers or electrodes 28 (e.g., twelve electrodes) that, when exposed to the sample fluid, transmit a signal indicative of a particular constituent component of the sample fluid. The plurality of electrodes 28 can be provided with the transducer chip 14. In some embodiments, the transducer chip 14 can include any suitable number of transducers or electrodes 28. For example, the transducer chip 14 can include more than two, more than three, more than five, more than ten, or even greater number of transducers or electrodes 28. For example, the transducer chip 14 can include three to fifteen electrodes, six to fifteen electrodes, or ten to fifteen electrodes. A fluid pathway 30 can extend and circulate over the electrodes 28 to expose the electrodes 28 to the sample fluid. The electrodes 28 can electrically connect to corresponding I/O pads.

During a treatment procedure, the sensor module 10 can operate in a plurality of modes. The one or more valves 22 can have a plurality of positions configured to place the sensor module 10 in the plurality of modes. In some embodiments, the valve 22 can comprise a multi-way (e.g., three-way) valve that has a closed state that blocks a fluid to enter a sensor channel 36, a first open state that provides fluid communication between the sensor channel 36 and a calibration reservoir 32 that stores a calibration fluid, and a second open state that provides fluid communication between the sensor channel 36 and the fluid inlet (IN). For example, in a bypass mode, the valve 22 can be placed in the bypass position. For example, a valve motor 20 disposed in the reader 12 can connect to the valve 22 by way of a valve connector (e.g., a valve opening) configured to operably connect to a motor shaft of the valve motor 20. Processing electronics 24 in the reader 12 can be configured to send instructions to the valve motor 20 to place the sensor module 10 in the bypass mode by, e.g., rotating the valve body to the bypass position. A sample fluid (such as the patient's blood, peritoneal dialysate, etc.) can enter a housing of the sensor module 10 by way of a fluid inlet (IN). In the bypass mode, the valve 22 can directly connect (without routing to the transducer chip 14) the fluid inlet (IN) to a fluid outlet (OUT) to convey the sample fluid outside the housing through a bypass pathway 48. In the bypass mode, therefore, the sample fluid (e.g., the patient's blood, peritoneal dialysate, etc.) may not be monitored by the sensor module, but may instead be recirculated to the sensor system 1.

During a calibration mode, the valve motor 20 can place the valve 22 in the calibration position. Calibration liquid or fluid (also referred to as a quality control fluid, or QC fluid, e.g., a biocompatible fluid such as water, saline, etc.) can be provided in a calibration reservoir 32 of the housing. In some embodiments, the calibration fluid can comprise water with known concentrations of species (e.g., sodium, potassium, pH, calcium, etc.). In the calibration position, as in the bypass position, the sample fluid can pass directly from the fluid inlet (IN) to the fluid outlet (OUT) to bypass the sensing assembly. The valve 22 can fluidly connect a calibration channel 34 that is connected to the calibration reservoir 32 to a sample entry channel or a sensor channel 36 in the fluid pathway 30 and the sensing assembly. The calibration fluid and the calibration mode can serve to reset the sensor module 10 by flushing the sensing assembly of older sample fluid and/or other debris. Thus, the calibration fluid may also be deemed a purge fluid. The processing electronics 24 can be configured to recognize that the sample fluid is in fluid communication with each of the bare or functionalized electrodes 28.

In some embodiments, the calibration fluid can exit the fluid pathway 30 and the sensing assembly (e.g., the transducer chip 14) by way of a sample exit channel 38 and/or the fluid outlet (OUT). In some embodiments, the calibration fluid can be entrained with the sample fluid and recirculated into the patient's body. In other embodiments, during calibration, the calibration fluid can be redirected to a separate waste container 40, which may be disposed of and may not be circulated into the patient's body.

During a sensing mode, one or more valves 22 can open/close to allow the fluid to enter a sensing channel (e.g., the sensor channel 36). In some embodiments, the valves 22 can be controlled by a control module (e.g., the controller 18) that is coupled to the sensor module 10 in the reader 12. The sample fluid can interact with the electrodes 28 and, in response, the electrodes 28 can transmit a signal to the I/O pads and the leads of the reader 12 indicative of respective constituent components of the sample fluid. The processing electronics 24 of the reader 12 or the computing device 26 in communication with the reader 12 can determine an amount of each constituent component detected by the sensor module 10. When the amount exceeds or is below a threshold for that component, the processing electronics 24 can be configured to send an alert to the clinician and/or modify the treatment procedure (e.g., shut off the procedure, change the parameters of the procedure, etc.), in some embodiments.

The processing electronics 24 of the reader 12 can be programmed to automatically switch between various modes of the sensor module 10. For example, the process electronics 24 can be programmed to automatically cycle between the sensing mode (in which the constituent component(s) of the sample fluid are monitored) and the calibration mode (in which the calibration fluid flushes the sensing assembly). In some embodiments, the processing electronics 24 can be further configured to automatically switch into the bypass mode when the sensing device (e.g., the transducer chip 14) is to be inactive. In some embodiments, the user (e.g., patient or clinician) can manually switch modes by engaging a user interface (UI) 42 of the reader 12. The UI 42 of the reader 12 can comprise a touch screen and/or buttons that enable the user and/or clinician to interact with the reader 12. In some embodiments, the UI 42 can include a display that indicates the levels of the constituent components in the fluids.

In addition, the sensor module 10 can include a fill mode. In the fill mode, the calibration fluid can be pumped or otherwise driven into the fluid inlet (IN). In some embodiments, the flow of the calibration fluid can be controlled by the processing electronics 24 of the reader 12. The valve 22 can be placed in the fill position (e.g., manually or by the motor), and the calibration fluid can be transferred from the fluid inlet (IN) to the calibration reservoir 32 by way of the calibration channel 34. Thus, in the fill mode, the calibration fluid can travel in a reverse direction along the calibration channel 34 as compared to during the calibration mode. In various embodiments, the calibration reservoir 32 can be filled prior to use, e.g., in the factory or assembly plant. The fill mode can serve as wetting/hydration step at the start of the sensor operation.

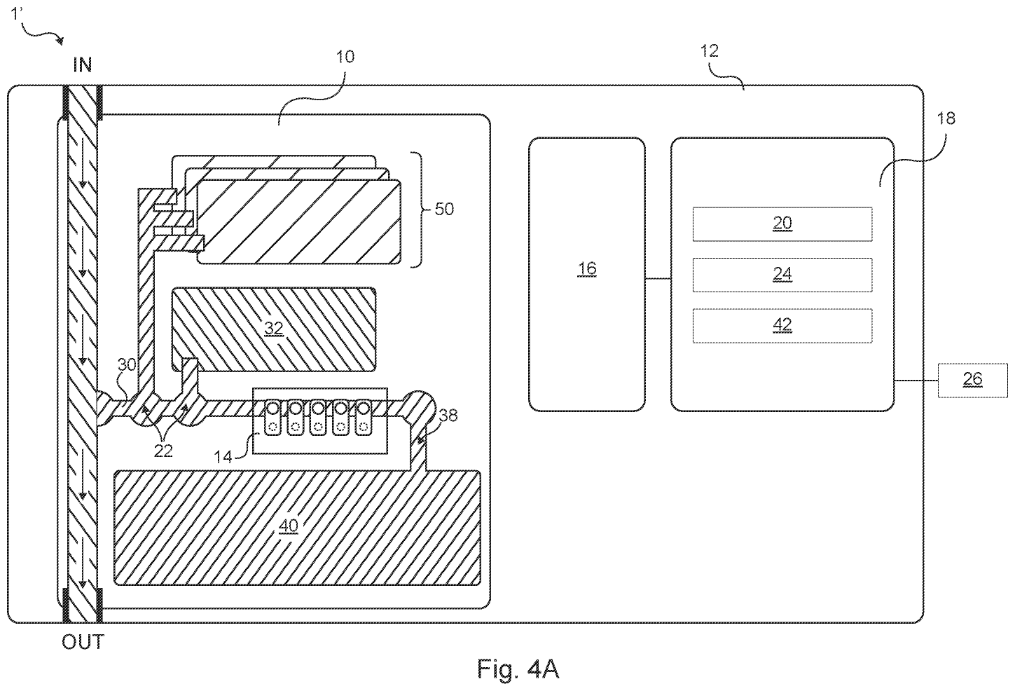

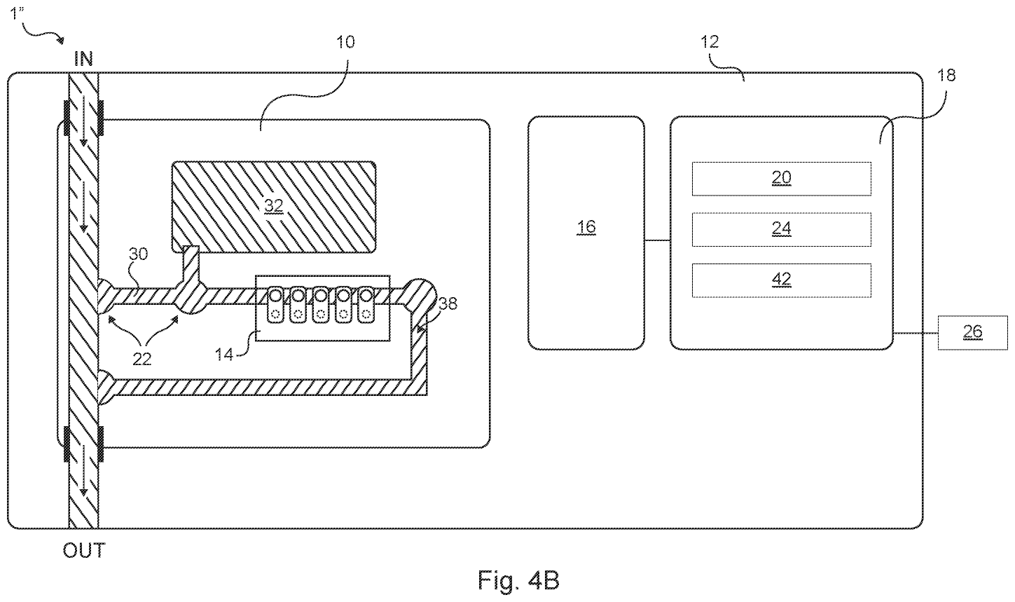

In addition, the sensor module 10 may include multiple calibration fluid compartments and/or quality control liquid(s) compartment(s) as shown in FIG. 4A.

Beneficially, the sensor system 1 (e.g., the fluid system) disclosed herein can enable the patient to conduct medical treatments (such as dialysis) at home, or otherwise outside of a clinical setting. As one example, when the patient goes to bed for the night, the patient can initiate a dialysis system as an example of the sensor system 1, and connect a fluid sensor module as an example of the sensor module 10 to the reader 12. The fluid inlet (IN) and fluid outlet (OUT) of the fluid sensor module can fluidly connect to the treatment system of the medical device, for example, by way of a Luer lock or other fluid coupler. The dialysis (or other) machine can be activated, and the fluid sensor module can automatically cycle between bypass mode, calibration mode, and sensing mode (in any suitable order). When an anomaly is detected during the sensing mode, the reader 12 can transmit an alarm to the clinician and/or otherwise modify the treatment procedure automatically. Once the treatment procedure is completed, the patient can disengage the treatment system, and remove the fluid sensor module. The fluid sensor module can be disposed and, for the next treatment procedure, a new fluid sensor module can be inserted into the reader 12 and connected to the medical device.

A significant number of medical procedures can be accompanied by a desire to monitor the concentrations of species in patient's physiological fluids (e.g., blood, urine, sweat) and in fluids that are used for treatment (e.g., dialysate infusion and effluent during kidney dialysis treatment). Accurate measurements today are complicated by the need to use either remote laboratory analysis instruments (so-called blood gas analysis (BGA) instruments) or point of care (PoC) single use diagnostic devices, with both needing extra steps of collecting samples first and then performing the analysis. This puts an extra burden to the physician/caregivers and may result in human errors. Existing in-line monitoring solutions are limited in either functionality (only few parameters can be measured) and/or not cost-competitive to “collection+remote analysis” approach. The present disclosure provides a novel solution that resolves these drawbacks. For example, the sensor system 1 disclosed herein can perform in-line monitoring without these drawbacks. The sensor system 1 disclosed herein includes a sensor module 10 that can comprise a disposable after every treatment (that can be between minutes and several days) cartridge incorporating sensor elements (e.g., the electrode chip 14) as well as fluidics components 46 (e.g., valves, analyte sampling and calibration actuation interfaces, etc.) to sample the fluid of interest (while providing uninterrupted flow of such fluid not to affect the treatment); re-calibrate the sensors before and/or after every sample by exposing the sensor elements to the calibration fluid with known concentrations of species of interest from container of calibration fluid held in the cartridge (e.g., the calibration reservoir 32); protect the extracorporeal fluid from any interaction with the sensors or calibration fluid by collecting the samples and used calibration fluid into the waste compartment 40, also held in the cartridge; fluidics actuation/control, electronic and optical interface to sensors and user communications are performed by durable part of the system (e.g., a reader 12) that accepts and operates the disposable cartridge at every treatment. For example, the durable part of the system can be permanently interfaced to standardized treatment equipment/infrastructure to facilitate treatment decisions by the physicians/caregiver. In some applications, the disposable sensor cartridge can enable up to 100 accurate measurements of the species of interest per therapeutic treatment with typically-10 species being measured at the same time. The sensor elements can be of electrochemical type (e.g., impedimetric, potentiometric, or amperometric for ions and metabolites sensing) or optical type (e.g., absorption/transmission, colorimetric for cell and biomarkers identification and measurements). Examples of the sensors could be ion sensors (both anions and cations such as Na+, K+, Ca++, pH, Mag++, Cl—, NH3-), metabolite sensors (e.g., Creatinine, Glucose, Urea), dissolved gasses sensors (e.g., pO2, pCO2), biomarker sensors (e.g., IL-6, MMP, and in general cytokines) and also configure to function as reference potential and counter electrodes (to enable accurate potentiometric and amperometric sensors measurements). The transducer chip 14 can include analog electrical and/or optical interfaces. The interface unit 16 and the controller 18 can be compatible to electronic and/or optical read-out.

FIG. 4A is a system outline of a sensor system 1′ according to an embodiment. The sensor system 1′ can be generally similar to the sensor system 1 of FIG. 3. In the sensor system 1′ the sensor module 10 includes one or more compartments 50 such as multiple calibration fluid compartments and/or quality control (QC) liquid(s) compartment(s). The valve 22 can be controlled to let the fluid in the one or more compartments 50 flow in a portion of the fluid pathway 30 and to the transducer chip 14. In the illustrated embodiment, the calibration reservoir 32 and the one or more compartments 50 are connected to the fluid pathway 30 by different valves 22. However, the calibration reservoir 32 and the one or more compartments 50 may be connected to the fluid pathway 30 by the same valve 22 in some embodiments.

The one or more compartments 50 may store different calibration fluids. For example, each compartment of the one or more compartments 50 can be configured to each store a different calibration fluid with known quantities of one or more constituents to be sensed that is suitable for a particular sensor (such as ion sensors, metabolite sensors, dissolved gasses sensors, or biomarker sensors) implemented by a sensing element (e.g., the transducer chip 14). The calibration liquid or fluid can comprise, for example, water with known concentrations of species (e.g., sodium, potassium, pH, calcium, etc.) to calibrate the sensors. For example, the sensor module can include a first calibration compartment (e.g., the calibration reservoir) that is configured to store a first calibration fluid, and a second calibration compartment that is configured to store a second calibration fluid.

In some embodiments, the calibration reservoir 32 can be part of the one or more compartments 50. In some embodiments, each compartment of the one or more compartments 50 can have a valve. In some other embodiments, a multi-way valve can control the flow of the fluids stored in the one or more compartments 50. In some embodiments, a mixture of two or more fluids from the one or more compartments 50 can be provided to the sensor channel 36 and to the transducer chip 14.

FIG. 4B is a system outline of a sensor system 1″ according to an embodiment. The sensor system 1″ can be generally similar to the sensor system 1 of FIG. 3. In the sensor system 11″ of FIG. 4B, the waste container 40 present in the sensor system 1 of FIG. 3 is omitted. In some embodiments, the calibration fluid can exit the fluid pathway 30 and the sensing assembly (e.g., the transducer chip 14) by way of a sample exit channel 38 and the fluid outlet (OUT), and the calibration fluid can be entrained with the sample fluid and recirculated into the patient's body.

In some embodiments, the embodiments of FIGS. 3, 4A, and 4B can be combined. For example, sample exit channel 38 of a sensor system may be connected to a waste compartment 40 and to the outlet (OUT). In such embodiments, a valve may selectively rout certain fluid to the waste compartment 40 and different fluid to the outlet (OUT). For example, a sample fluid that enters into the sensor module 10 from the inlet (IN) can be sensed by the transducer chip 14 and be directed to the outlet (OUT), and a calibration fluid stored in the calibration reservoir 32 can be directed to the waste compartment after it has passed through the transducer chip 14.

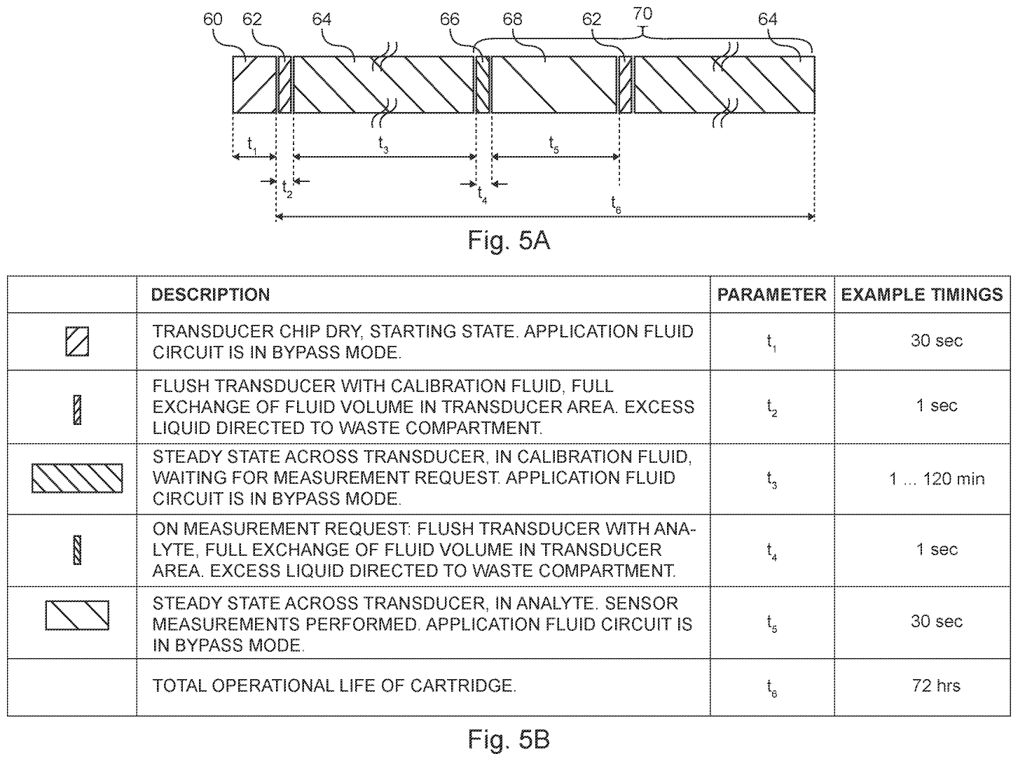

FIG. 5A shows a timing diagram of operating a sensor module of a sensor system, according to an embodiment. FIG. 5B is a chart showing example timings of various states in the operation of the sensor module. In some embodiments, the operation of the sensor module (e.g., the sensor module 10) can be controlled at least in part by the reader 12. In a first state 60, the sensing element or the electrodes of the transducer chip 14 can be dry and free from a liquid. In the first state 60, the sensor module 10 can be in a bypass mode in which the sample fluid does not enter the sensor channel 36. In some applications, a duration t1 of the first state 60 can be about 30 seconds. For example, the duration t1 of the first state 60 can be 5 seconds to 10 minutes, 10 seconds to 5 minutes, or 20 seconds to 1 minute.

A second state 62 can follow the first state 60. In the second state 62, the electrodes of the transducer chip 14 can be flushed with a calibration fluid through the sensor channel 36. In some applications, a duration t2 of the second state 62 can be about 1 second. For example, the duration t2 of the second state 62 can be 0.5 seconds to 5 seconds, 0.5 seconds to 3 seconds, or 0.75 seconds to 3 seconds.

A third state 64 can follow the second state 62. In the third state 64, the calibration fluid can stay in the sensor channel 36 until a measurement request is received. In the third state 64, the sensor module 10 can be in the bypass mode. In some applications, a duration t3 of the third state 64 can be about 1 minute to 120 minutes. For example, the duration t3 of the third state 64 can be 1 minute to 60 minutes, 30 minutes to 120 minutes, or 10 minutes to 60 minutes.

A fourth state 66 can follow the third state 64. In the fourth state 66, the sample fluid or analyte can be provided. For example, the calibration fluid in the sensor channel 36 can be flushed and replaced with the sample fluid. In some embodiments, an excess fluid can be directed to the waste compartment 40. In some applications, a duration t4 of the fourth state 66 can be about 1 second. For example, the duration t4 of the fourth state 66 can be 0.5 seconds to 5 seconds, 0.5 seconds to 3 seconds, or 0.75 seconds to 3 seconds.

A fifth state 68 can follow the fourth state 66. In the fifth state 68, the sample fluid can stay in the sensor channel 36 and interact with the electrodes of the transducer chip 14 for measurement. In the fifth state 68, the sensor module 10 can be in the bypass mode. In some applications, a duration t5 of the fifth state 68 can be about 30 seconds. For example, the duration t5 of the fifth state 68 can be 10 seconds to 1 minute, 15 seconds to 45 seconds, or 20 seconds to 40 seconds.

After the fifth state 68, a cleaning process (the second and third states 62, 64) can follow to clean the sensor channel 36 and surfaces of the electrodes of the transducer chip 14. After cleaning the sensor channel 36 and the surfaces of the electrodes, another measurement can take place upon request and repeat the measurement process (the fourth and fifth states 66, 68). The cleaning and measurement cycle 70 can repeat a number of times. The total number of times (N times) of the cleaning and measurement cycle 70 can be determined based at least in part on a size of the calibration reservoir 32, a size of the one or more compartments 50, and/or a size of the waste compartment 40. In some embodiments, the total operational life of the sensor module 10 can be about 72 hours. For example, the total operational life of the sensor module 10 can be in a range between 24 hours and 148 hours, 36 hours and 124 hours, or 48 hours and 100 hours.

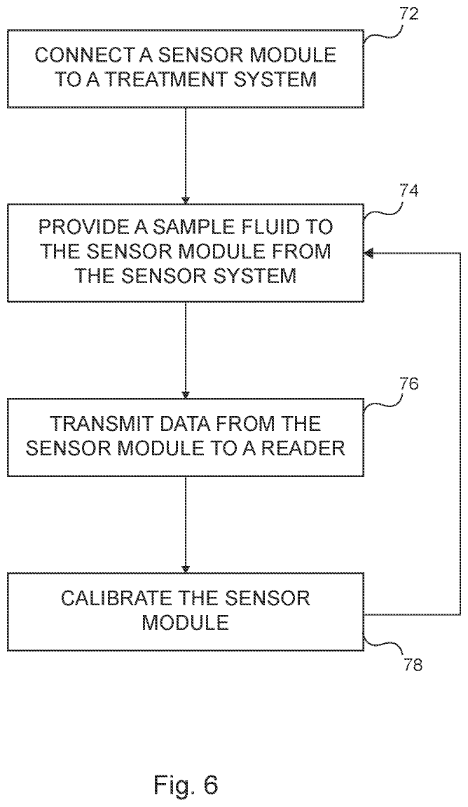

FIG. 6 is a flow chart showing a method of monitoring a sample fluid. In block 72, a sensor module is connected to a treatment system. The treatment system can be a medical device, such as a dialysis treatment system. The sensor module can be connected in-line to the treatment system. In block 74, the sample fluid can be provided to a sensing element (e.g., the transducer chip 10) of the sensor module from the treatment system. The sample fluid can interact with the sensing element for measurement. In block 76, the sensor module can transmit a signal indicative of a particular constituent component of the sample fluid to a reader connected to the sensor module. The reader can comprise processing electronics that are configured to control the operation of sensor module. In block 78, the sensor module can be calibrated. After the calibration, the process can return to block 74 for further measurements. The processes for measuring the constituent component of the sample fluid disclosed herein, such as those with respect to FIGS. 5A and 5B can be implemented in blocks 74, 78 of FIG. 6.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Moreover, as used herein, when a first element is described as being “on” or “over” a second element, the first element may be directly on or over the second element, such that the first and second elements directly contact, or the first element may be indirectly on or over the second element such that one or more elements intervene between the first and second elements. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

While certain embodiments have been described, these embodiments have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while blocks are presented in a given arrangement, alternative embodiments may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various embodiments described above can be combined to provide further embodiments. The accompanying claims and their equivalents are intended to cover such forms or modifications as would fall within the scope and spirit of the disclosure.

What is claimed is:

1. A fluid sensor system configured to perform in-line monitoring, the fluid sensor system comprising:
a sensor module comprising a sensing channel configured to receive a sample fluid, two or more calibration compartments, and a sensing element configured to interact with the sample fluid in the sensing channel; and
a reader electrically and mechanically coupled to the sensor module, the reader comprising a controller configured to control operation of the fluid sensor system,
wherein the fluid sensor system is configured to connect in-line with a treatment system.

2. The fluid sensor system of claim 1, wherein the sensing element comprises a plurality of transducers.

3. The fluid sensor system of claim 2, wherein the transducers are configured to interact with the sample fluid and transmit a signal indicative of a particular constituent component of the sample fluid.

4. The fluid sensor system of claim 3, wherein the sensing element comprises at least three transducers.

5. The fluid sensor system of claim 1, wherein the two or more calibration compartments comprises a first calibration compartment configured to store a first calibration fluid.

6. The fluid sensor system of claim 5, wherein the two or more calibration compartments comprises a second calibration compartment configured to store a second calibration fluid different from the first calibration fluid.

7. The fluid sensor system of claim 1, wherein the reader is connectible to an external device and configured to send measurement results to the external device.

8. The fluid sensor system of claim 1, further comprising a fluid pathway having a fluid inlet and a fluid outlet, the sensing channel is connected to a portion of the fluid pathway between the fluid inlet and the fluid outlet.

9. The fluid sensor system of claim 8, further comprising a valve between the sensing channel and the fluid pathway, the controller is configured to control the valve to open or close.

10. The fluid sensor system of claim 8, wherein an exit channel downstream of the sensing element is connected to the fluid pathway.

11. The fluid sensor system of claim 8, wherein the fluid inlet or the fluid outlet comprises a Luer lock configured to couple to a treatment system.

12. The fluid sensor system of claim 1, further comprising a waste compartment positioned downstream of and connected to the sensing element by way of an exit channel.

13. The fluid sensor system of claim 1, wherein a sample exit channel downstream of the sensing element is connected to the treatment system through a fluid outlet of the fluid sensor system.

14. A method of monitoring a sample fluid, the method comprising:
connecting a sensor module in-line with a treatment system;
providing the sample fluid from the treatment system to a sensing element to thereby cause the sample fluid to interact with the sensing element;
transmitting a signal indicative of a particular constituent component of the sample fluid by the sensing element to a reader electrically and mechanically coupled to the sensor module; and
calibrating the sensing element using a calibration fluid stored in two or more calibration compartments.

15. The method of claim 14, wherein a fluid inlet and a fluid outlet of the sensor module connect to the treatment system by way of a Luer lock.

16. The method of claim 14, wherein the sensing element comprises a plurality of transducers.

17. The method of claim 14, wherein the calibration fluid is stored in a first calibration compartment of the two or more calibration compartments, and a second calibration fluid is stored in a second calibration compartment of the two or more calibration compartments.

18. The method of claim 14, wherein the sensor module comprises a waste compartment positioned downstream of the sensing element and configured to receive the sample fluid that has passed through the sensing element.

19. The method of claim 14, further comprising calibrating the sensor module after providing the sample fluid to the sensing element.

20. The method of claim 19, further comprising providing a second sample fluid from the treatment system to the sensing element to thereby interact the second sample fluid with the sensing element.
